(19) United States
(12) Patent Application Publication
(10) Pub. No.: US 2026/0248627 A1
(43) Pub. Date: Aug. 27, 2026

(54) STENT SYSTEM

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); AMIRHAMZAH MAHMADIQBAL SHAIKH, Valsad (IN); YASHKUMAR BIPINBHAI BHANDARI, Valsad (IN)

(73) Assignee: MERIL LIFE SCIENCES PVT LTD, Vapi, GJ (IN)

(21) Appl. No.: 18/872,444

(22) PCT Filed: Mar. 28, 2023
(86) PCT No.: PCT/IN2023/050296

§ 371 Date: Dec. 6, 2024

(30) Foreign Application Priority Data
Jun. 16, 2022 (IN) 202221034628

(51) Int. Cl. A61F 2/90; A61F 2/848

(52) U.S. Cl. CPC A61F 2/90; A61F 2002/8486; A61F 2250/0039; A61F 2250/0098

(57) ABSTRACT

The present invention discloses a stent. A proximal most ring is disposed at a proximal end and a distal most ring is disposed at a distal end. A plurality of rings extends between the proximal ring and the distal ring. A first link couples a crown of one of the proximal most ring or the distal most ring to a respective adjacent crown of an adjacent ring. A second link couples a crown of a ring to a respective adjacent crown of an adjacent ring. A plurality of rows open cells is flanked by two or more rows of closed cells. A plurality of radiopaque markers coupled to the proximal most ring are circumferentially offset from a plurality of markers coupled to the distal most ring. The proximal most ring and the distal most ring of the stent are configured to flare by a pre-defined angle (x).

### FIELD OF INVENTION

The present invention relates to a medical device. More specifically, the present invention relates to a stent system.

### BACKGROUND

Peripheral vascular disease is characterized by deposits of fats, calcium, and plaques on inner walls of arteries. These deposits damage the artery and vein tissues, and impede their flexibility, resulting in narrow blood vessels with reduced blood flow. These deposits can partially or completely block arteries over time, causing problems throughout the body leading to risk of being diagnosed with diabetes, hypertension, and other diseases.

For the treatment of the aforementioned problems, a stent is used to enlarge these blocked/narrow segments of the artery to improve blood flow. Stent is a cylindrically shaped device that provides excellent radial and mechanical strength to the blocked blood vessels.

A stent with desirable property includes long length with low thickness and good mechanical strength. Ideally, the stent should allow good flow of blood and mimic the mechanical properties of a natural artery. It should be flexible with good kink resistance, facilitate various natural functions of the veins and arteries, allow blood to return from the periphery to the heart, balance blood volume and pressure between the various organs of the body and mimic the natural artery.

However, conventional stents fail to address the above as fabrication of such a stent is extremely difficult. During fabrication of such stent, it becomes very bulky with increase in lengths. Further, with increasing lengths of tapered implantation site (vasculature), there is a high risk of stent displacement and migration from the implantation site post deployment of the stent.

Therefore, such stent profiles become very complex to manufacture due to their bulky structure. Further, it becomes necessary to change the existing design of the stent to improve their mechanical strength for better flexibility and improved radial strength especially for long length arteries having scattered blockages.

Moreover, existing stents fails to preserve their integrity (mechanical properties and/or durability) when deployed in complex anatomy. This may result in severe post-implantation complication. This phenomenon is more commonly observed in stent implants deployed in long stenosed vasculature having non-linear anatomy.

To overcome the said limitations, there is a need for a stent which is flexible and kink resistant with optimum radial forces for long length arteries.

### SUMMARY

The present invention relates to a stent to be deployed in a lumen with reduced risk of displacement. The stent includes a proximal end and a distal end. A proximal most ring is disposed at the proximal end and a distal most ring is disposed at the distal end. The proximal most ring and the distal most ring include a plurality of first struts connected to each other in a predefined pattern forming a crown at each connection. A plurality of rings extends coaxially between the proximal most ring and the distal most ring. Each of the plurality of rings include a plurality of second struts connected to each other in the predefined pattern forming the crown at each connection. A first link couples a crown of one of the proximal most ring or the distal most ring to a respective adjacent crown of an adjacent ring. A second link couples a crown of a ring to a respective adjacent crown of an adjacent ring. Two or more rows of closed cells are disposed at each of the proximal end and the distal end respectively. Each row of closed cells are formed by coupling each crown formed by at least one of the plurality of second struts to a respective adjacent crown formed by at least one of the plurality of first struts or the plurality of second struts via at least one of the first links or the second links. A plurality of rows open cells is flanked by two or more rows of closed cells. Each row of open cells are formed by selectively coupling a crown formed by at least one of the plurality of second struts to a respective adjacent crown formed by the plurality of second struts via the second links. The second links of the plurality of row of open cells are arranged in a predefined pattern. A plurality of radiopaque markers coupled to at least one of the crowns of the proximal most ring and the distal most ring. The plurality of radiopaque markers coupled to the proximal most ring are circumferentially offset from the plurality of radiopaque markers coupled to the distal most ring. The plurality of first struts is longer than the plurality of second struts. The first link is offset inwardly to enable the plurality of first struts of the proximal most ring and the distal most ring of the stent to flare by a pre-defined angle relative to a central axis at the proximal end and the distal end respectively, in an expanded state of the stent, thereby reducing risk of any displacement when the stent is deployed in a lumen.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts a lateral view of a stent 100 in expanded state in accordance with an embodiment of the present invention.

FIG. 1a depicts a portion of the stent 100 in accordance with an embodiment of the present invention.

FIG. 1b depicts an axial view of the stent 100 in accordance with an embodiment of the present invention.

FIG. 2 depicts a lateral view of the stent 100 in collapsed state in accordance with an embodiment of the present invention.

FIG. 3 depicts a stent 100 with S-shaped links 170 in expanded state in accordance with an embodiment of the present invention.

FIG. 4 depicts a tapered stent 600 in accordance with an embodiment of the present invention.

FIG. 5 depicts a mandrel 10 in accordance with an embodiment of the present invention.

FIG. 5a depicts a method 700 to make the tapered stent 600 in accordance with an embodiment of the present invention.

FIG. 6 depicts a delivery system 20 in accordance with an embodiment of the present invention.

FIG. 6a depicts a method 800 to load the stent 100 onto the delivery system 20 in accordance with an embodiment of the present invention.

FIG. 6b depicts a method 900 to deploy the stent 100 in accordance with an embodiment of the present invention.

FIG. 7 depicts a C-shape anatomy of the stent 100 post implantation at a target site in accordance with an embodiment of the present invention.

FIG. 7a depicts an S-shape anatomy of the stent 100 post implantation at the target site in accordance with an embodiment of the present invention.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a stent system (or stent) is disclosed. The stent may be deployed in a target site for treatment. The target site may include without limitation, a peripheral and/or a coronary artery or any other lumen of uniform or varying diameter. The stent helps to improve luminal diameter of the target site by removing plaque (or any other obstruction), thereby, restoring blood flow at the target site. The stent alleviates conditions like atherosclerosis of peripheral arteries which result in hardening of arteries. The stent can be either self-expandable or balloon expandable from a collapsed state to an expanded state. In an exemplary embodiment, the stent is self-expandable.

The stent of the present invention includes a plurality of first struts and a plurality of second struts. The plurality of first struts forms a proximal most ring and a distal most ring. The plurality of second struts form a plurality of rings disposed between the proximal most ring and the distal most ring.

The proximal most ring, the distal most ring and the plurality of rings connect with each other to form a hybrid cell structure. The said structure includes two or more rows of closed cells both at the proximal end and the distal end of the stent while open cells extend between the closed cells.

A plurality of first links is used for connecting first struts of the proximal most ring and/or the distal most ring to second struts of respective ring adjacent to the proximal most ring and the distal most ring to form a row of closed cells at a proximal end and a distal end of the stent respectively. Further, a plurality of second links is used for connecting the plurality of rings to form a row of closed cells and/or a row of open cells between two adjacent rings.

The plurality of first links facilitates the first struts to be longer than the second struts. The first links along with longer first struts facilitates flaring of the proximal end and the distal end of the stent in the expanded state. The flared proximal and distal ends help to reduce risk of displacement of the stent during and after the deployment of the stent. Hence, the flaring of the proximal and distal ends increases the resistance to migration and enhances the holding capacity of the stent.

The hybrid cell structure of the stent reduces micro-cracks formed due to uneven loadings and forces acting on it, thereby resulting in a fracture proof stent. The hybrid cell structure of the stent further helps in better flexibility and track-ability into tortuous anatomy during the deployment of the stent. The stent provides excellent radial strength and flexibility without kinking, thereby making it suitable to be used in both short as well as long peripheral or coronary arteries. Due to kink resistance, these stents are well suited for the tortuous vessel pathways of peripheral arteries.

Further, the stent may include a tapered profile that provides excellent radial strength and supports the target (or implantation) site without any displacement. The stent implanted at the target site results in improved luminal diameter, restoration of blood flow and physical support of narrowed peripheral vessels. Moreover, the stent of the present invention can easily be advanced to the target site without any mechanical failure such as bending, buckling, or kinking. The stent includes a reduced/low crimp diameter with increased strength and durability.

The excellent radial as well as mechanical strength of the stent makes the stent effective under continuous loading conditions where the blood vessels are subjected to radial and axial forces without kinking and reduces the risk of overlapping of struts of the stent. In other words, the stent can easily resist forces coming from any direction thereby withstand radial and axial fatigue.

The proximal end and the distal end of the stent includes one or more radiopaque markers that are coupled to alternate crowns such that the radiopaque markers of the proximal end are circumferentially offset from the radiopaque marker of the distal end. Such arrangement of the radiopaque markers in offset manner allows visualization of all the radiopaque markers provided on both the proximal end and the distal end of the stent, when viewed axially. This further helps to clearly visualize the complete internal periphery of the treatment site (such as a blood vessel) during fluoroscopy thus facilitating easy navigation of the stent within tortuous anatomy during implantation.

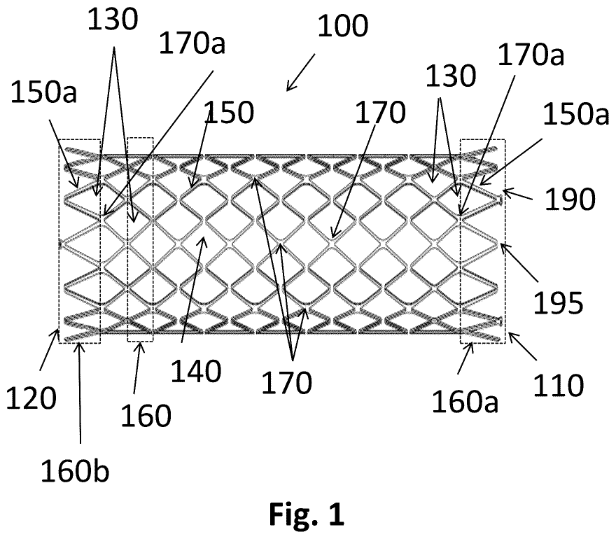

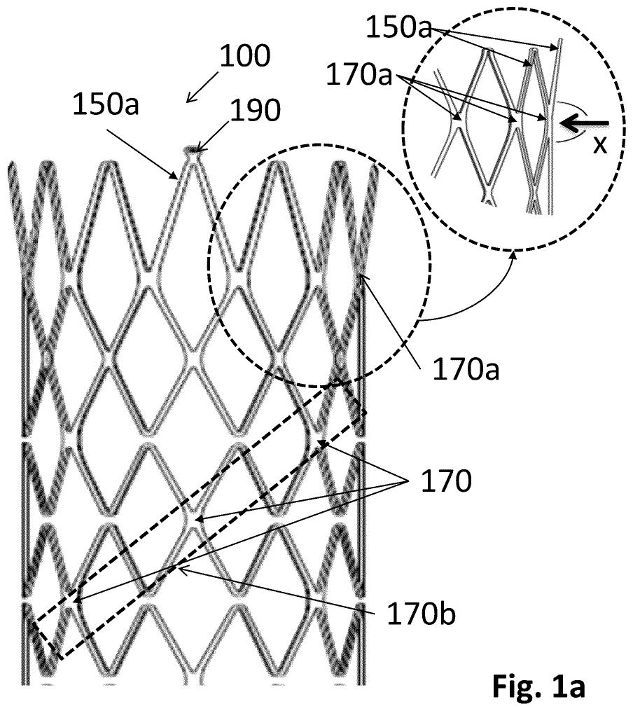

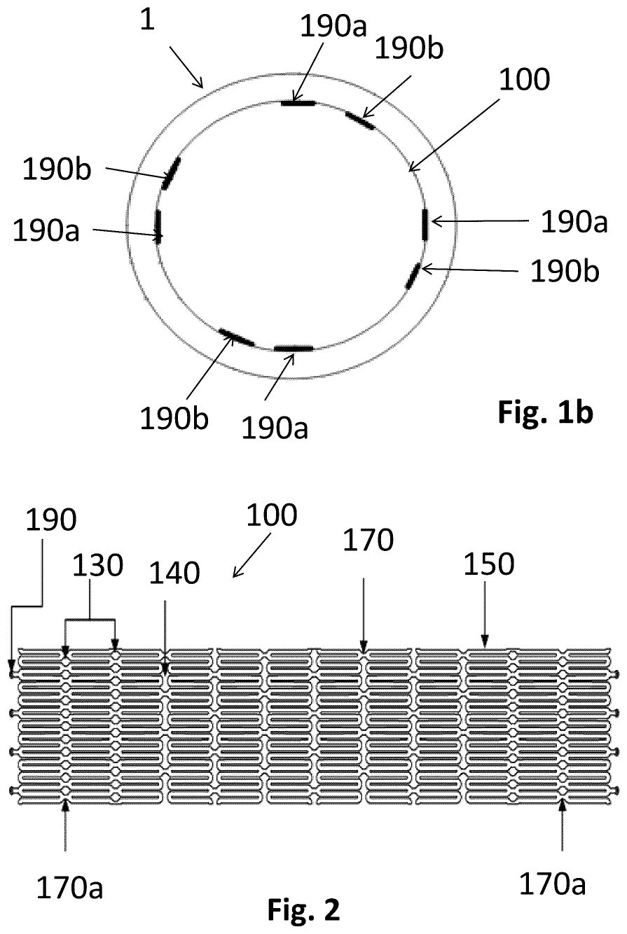

Now referring to figures, FIG. 1 shows a stent 100 of the present invention. The stent 100 includes a proximal end 110 and a distal end 120.

The stent 100 may be made of a material including, but not limited to stainless steel, nitinol, cobalt chromium alloy, etc. In an exemplary embodiment, a Nitinol tube (medical grade) is used to manufacture the stent 100. Nitinol tubes are considered as the best choice of material due to its unique properties and good clinical outcomes which perfectly satisfies the need in various stenting applications. Further, Nitinol is highly flexible and super elastic material that can be processed to maintain desired geometry of the stent 100.

The stent 100 may have a diameter ranging from 5 mm to 14 mm, more preferably 6 mm to 10 mm. The stent 100 may have a length ranging from 30 mm to 220 mm. The diameter and the length of the stent 100 changes based on application. In an exemplary embodiment, the diameter of the stent 100 remains uniform throughout the length of the stent 100.

The stent 100 of the present invention includes a hybrid cell structure (described below) which reduces micro-cracks formed due to uneven loadings and forces acting on it, thereby resulting in a fracture proof stent. In an exemplary embodiment, the hybrid cell structure of the stent 100 is formed by laser cutting the Nitinol tube which reduce/lowers the crimp profile of the stent 100 and chances of fracture during manufacturing of the stent 100. The hybrid cell structure of the stent 100 further helps in better flexibility and track-ability into tortuous anatomy during deployment of the stent 100.

The stent 100 may include an expanded state (as shown in FIG. 1) and a collapsed state (as shown in FIG. 2). This expanded state of the stent 100 helps in the deployment of the stent 100 at a target site with minimal effort and provides more grip to an inner lumen of the target site throughout the process of deployment. The stent 100 may be crimped to the collapsed state for easy and atraumatic navigation within the tortuous vasculature during deployment (described below). The stent 100 may attain the expanded state post deployment at the target site.

The stent 100 includes a proximal most ring 160a and a distal most ring 160b disposed at the proximal end 110 and distal end 120 respectively. The proximal most ring 160a and the distal most ring 160b may be made of a plurality of first struts 150a. A plurality of circumferential rings 160 (or rings 160) extends between the proximal most ring 160a and the distal most ring 160b. The rings 160 may be coaxially aligned with each other. The rings 160 may be made of a plurality of second struts 150.

The adjacent rings 160 (or rings 160 adjacent to each other) may have a gap of a pre-defined length between them. The predefined length may range from 0.2 mm to 0.3 mm. The pre-defined length between the adjacent rings 160 renders the stent 100 tangle free. Hence, the rings 160 are spaced at sufficient distance so that it does not get tangled with each other during the positioning of stent 100 inside the patient.

The rings 160 are formed by connecting a plurality of second struts 150. The second struts 150 may have a wall thickness ranging from 80 μm to 240 μm. The wall thickness may be defined as a thickness of the stent 100 in a radial direction. The said range of wall thickness of the second struts 150 provides low profile to the stent 100 resulting in reduced wall (of the stent 100) to body (vessel area of the implantation site) ratio after implantation of the stent 100 at the target site. The second struts 150 may have a length ranging from 1 mm to 8 mm. The second struts 150 may have a width ranging from 60 μm to 180 μm. The said range of width of the second struts 150 reduces risk of thrombus formation after implantation of the stent 100 at the target site.

The second struts 150 of the rings 160 may be connected to each other in a pre-defined pattern. In an exemplary embodiment, as shown in FIG. 1, the second struts 150 of the ring 160 are connected with each other in a zig-zag pattern to form a crown 195 at each connection. The stent 100 may have 12 to 18 crowns 195 depending on the diameter of the stent 100. Each crown 195 may resemble convex shape or concave shape depending upon perspective view. For example, a crown 195 of the proximal most ring 160a resembles convex shape when viewed from the proximal end 110 and resembles concave shape when viewed from distal end 120. At least one crown 195 formed by the second strut 150 of each of the adjacent rings 160 may be coupled to each other by a second link 170. In other words, the second link 170 may couple at least one crown 195 of a ring 160 to a respective adjacent crown 195 of an adjacent ring 160. The said coupling between rings 160 via the second links 170 provides strength to the structure of the stent 100. Further, the crowns 195 may be relatively wider (compared to the first struts 150a and the second struts 150) to minimize recoil and also to reduce the concentration of stress at joints (strut-strut joint and/or strut-link joint).

The second links 170 may have a pre-defined length ranging from 1 mm to 3 mm. The second links 170 may have a wall thickness ranging from 100 μm to 400 μm. The wall thickness may be defined as a thickness of the stent 100 in a radial direction. The second link 170 may have a width ranging from 0.2 mm to 0.5 mm. The length of the second links 170 between each ring 160 may be similar throughout the length of the stent 100 which makes the structure of the stent 100 easier to analyze, design and manufacture.

The second links 170 may be straight, S-shaped, etc. In an exemplary embodiment, as depicted in FIG. 1, the second link 170 is straight. The straight second link 170 absorbs proper amount of radial force acting on the stent 100 without affecting its flexibility. The straight second link 170 also reduces the force required to deploy the stent 100 at the target site.

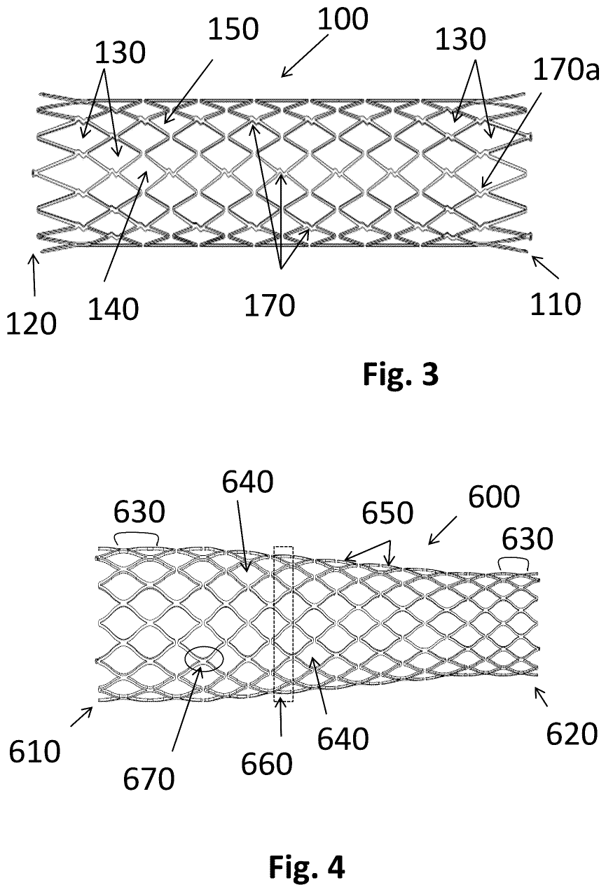

In another exemplary embodiment, as depicted in FIG. 3, the second link 170 is S-shaped. The S-shaped second links 170 provide high flexibility and excellent track-ability to the stent 100 in tortuous blood vessels. The S-shaped second link 170 also reduces the chances of strut fracture when the stent 100 is passed within the tortuous anatomy. It also provides high resistance against compression and bending stress towards the stent 100.

Coupling of the crowns 195 of the adjacent rings 160 via the second links 170 results in formation of a plurality of rows of closed cells 130 and/or open cells 140 (as shown in FIG. 1). The said connection between crowns 195 of the adjacent rings 160 provides strength and maintains flexibility, which further reduces stress concentration of second struts 150 at every crown 195.

Therefore, as described above, the stent 100 of the present invention includes a hybrid cell structure made of the closed cells 130 and the open cells 140. Due to the hybrid cell structure, the stent 100 of the present invention provides excellent balance between flexibility, conformability and kink resistance. It also provides high radial strength and optimum flexibility for the treatment of vasculature defects. The stent 100 offers optimal radial force without compromising flexibility. The hybrid cell structure of the stent 100 allows it to maintain a low crimp profile which can withstand different types of forces acting on stent 100 post deployment.

The row of closed cells 130 may be formed by coupling each crown 195 formed by at least one of the plurality of second struts 150 to a respective adjacent crown 195 formed by at least one of the plurality of second struts 150 of an adjacent ring 160 via the second links 170. The row of open cells 140 may be formed by selectively coupling a crown 195 formed by at least one of the plurality of second struts 150 to a respective adjacent crown 195 formed by the plurality of second struts 150 via the second links 170. In other words, the rows of open cells 140 may include one or more crowns 195 of a ring 160 not coupled to any adjacent crown 195 of an adjacent ring 160 via second links 170.

The stent 100 may include two or more rows of closed cells 130 at each of the proximal end 110 and the distal end 120 such that the rows of open cells 140 are flanked by the rows of closed cells 130. In other words, the rows of open cells 140 are disposed in between the rows of closed cells 130. In an exemplary embodiment, as depicted in FIG. 1, the proximal end 110 and the distal end 120 includes two rows of closed cells 130 each. The two rows of closed cells 130 do not allow the stent 100 to migrate from the target site during and after the deployment procedure and provides high radial strength and optimum flexibility for the treatment of vasculature defects. Further, the two rows of closed cells 130 at both the proximal end 110 and the distal end 120 of the stent 100 provide optimum radial force to inner lumen of the vessel which helps to maintain the structure throughout the lesion. It also provides better grip and reduce the chances of misalignment of stent 100 from the target site during deployment and also during physical movement of the vessels after deployment of the stent 100.

The open cells 140 and the closed cells 130 may have a pre-defined shape when the stent 100 is in expanded state. The shape of the open cells 140 and closed cells 130 may include, but not limited to elliptical, rectangular, diamond or hexagonal. In an exemplary embodiment, the stent 100 is made up of non-hexagonal shaped open cells 140 and hexagonal shaped closed cells 130. The open cells 140 may have a cell length (measured in a circumferential direction of the stent 100) of 5 mm which varies depending on the number of second links 170 used between adjacent rings 160, as well as the diameter of the stent 100.

The number of second links 170 between adjacent rings 160 may change according to the diameter of stent 100, number of open cells 140 and closed cells 130 and its application. For a given diameter of the stent 100, the number of second links 170 influences balance between radial strength and flexibility of the stent 100. The stent 100 may include three to five second links 170 between adjacent rings 160 to make the rows of open cells 140. In an exemplary embodiment, the stent 100 having diameter of 5 mm includes four second links 170 between adjacent rings 160 to make the rows of open cells 140. The hybrid cell structure along with appropriate number of second links 170 between the adjacent rings 160 help to maintain the mechanical properties of the stent 100. In an alternate embodiment, the stent 100 have more than four second links 170 in order to increase the radial strength of stent 100 without affecting its flexibility. The hybrid cell structure of the stent 100 eliminates kinking and provides excellent track-ability in tortuous anatomy.

The second links 170 of the rows of open cells 140 may be arranged in a pre-defined pattern. In an exemplary embodiment, the second link 170 of adjacent rings 160 forming open cells 140 are in alignment with each other forming a symmetrical pattern. In another exemplary embodiment, as shown in FIG. 1a, each second link 170 of the rows of open cells 140 are circumferentially offset (when compared with second links 170 of adjacent rings 160) to form a staggered pattern 170b in the form of a spiral like helix format. The pre-defined pattern of the second links 170 of the rows of open cells 140 reduces overall profile of the stent 100, making it easy to handle. It further increases the mechanical strength and maintains the durability of the stent 100.

In an exemplary embodiment, as shown in FIG. 1a, a plurality of first links 170a disposed at the proximal end 110 and the distal end 120 are offset inwardly. In an expanded state of the stent 100, the said offset of the first links 170a may enable the proximal most ring 160a and the distal most ring 160b to flare by a pre-defined angle (or inflection angle) ‘x’ relative to a central axis (not shown) of the stent 100. The central axis of the stent 100 may be defined as an imaginary line passing through a center of the stent 100 along its entire length.

In other words, the first links 170a may be similar to the second links 170 except that the first links 170a are offset inwardly. Similarly, the first struts 150a may be similar to the second strut 150 except that the first strut 150a may be longer than the second strut 150. Hence, similar to the second struts 150, the first struts 150a of the proximal most ring 160a and the distal most ring 160b may be connected to each other in the predefined pattern to form crowns 195 at each connection. Similarly, the first links 170a may facilitate coupling of each crown 195 of the proximal most ring 160a and/or the distal most ring 160b to respective adjacent crowns 195 of adjacent rings 160 to yield a row of closed cells 130. In other words, a row of closed cells 130 may be formed by coupling each crown 195 formed by at least one of the plurality of second struts 150 to a respective adjacent crown 195 formed by the plurality of first struts 150a via the first links 170a.

The first links 170a along with first struts 150a facilitates flaring (as shown in FIG. 1) of the proximal end 110 and the distal end 120 of the stent 100 in the expanded state. The flared proximal and distal ends 110, 120 help to further reduce the risk of displacement of the stent 100 during and after the deployment process. As shown in FIG. 1a, the flaring of the proximal and distal ends 110, 120 may be defined by the pre-defined angle ‘x’. The pre-defined angle ‘x’ may vary from 160 to 180 degrees, more preferably from 165 to 175 degrees.

The stent 100 may include a plurality of radiopaque markers 190 disposed at each of the proximal end 110 and the distal end 120 of the stent 100. The radiopaque marker 190 may be laser welded to the crown(s) 195 of the proximal most ring 160a and the distal most ring 160b. In an exemplary embodiment, a diameter of the radiopaque marker 190 is 0.5 mm with a thickness of 170 microns. The radiopaque marker 190 may be made from a material having a high level of radiopacity as well as provide good biological compatibility with the human body. The material of the radiopaque marker 190 may include but not limited to platinum, tantalum, iridium, tungsten, gold, etc. In an exemplary embodiment, the radiopaque marker 190 is made from tantalum. Tantalum radiopaque markers 190 provide biocompatibility to human body and share similarities in electrochemical properties compared to the Nitinol stent 100. The radiopaque markers 190 facilitate visualization of the stent 100 under fluoroscopy, which further helps to guide the stent 100 to its target site in the patient.

In an exemplary embodiment, as shown in FIG. 1b, the proximal most ring 160a at the proximal end 110 and the distal most ring 160b at the distal end 120 of the stent 100 includes the radiopaque markers 190, namely a proximal radiopaque marker(s) 190a and a distal radiopaque marker(s) 190b respectively. The proximal and distal radiopaque markers 190a and 190b are coupled to alternate crowns 195 of the proximal most ring 160a and the distal most ring 160b such that they are circumferentially offset from each other. Such arrangement of the radiopaque markers 190 in offset manner allows visualization of all the radiopaque markers 190 provided on the stent 100. Further, it helps to visualize the complete internal periphery of a target site 1 (such as a blood vessel) during fluoroscopy thus facilitating easy navigation of the stent 100 within tortuous anatomy during implantation.

The stent 100 may be coated with a pre-defined coating material thus yielding one or more coating layer over the stent 100. The coating layer may have predefined properties like thickness, hardness, roughness, etc. The thickness of the coating layer may range from 2 μm to 8 μm, more preferably from 4 μm to 6 μm. The hardness of the coating layer may range from 2000 HV to 2800 HV, more preferably from 2300 HV to 2500 HV. The roughness of the coating layer may range from 0.03 μm to 0.07 μm, more preferably from 0.04 μm to 0.06 μm. The predefined properties of the coating layer help to attain even and unvarying coating of the pre-defined coating material throughout the length of the stent 100. The predefined coating material may include but not limited to an anti-proliferative drug, a thin layer of Titanium-Niobium Nitride (Ti-Nb N) or a combination thereof. The anti-proliferative drug may include but not limited to Sirolimus, Everolimus, Zotarolimus, Paclitaxel, etc.

In an exemplary embodiment, the stent 100 is coated with a thin layer of Titanium-Niobium Nitride (Ti-Nb N) which reduces the chances of corrosion and the formation of thrombus at lesion in patient's body. The coating of Titanium-Niobium Nitride (Ti-Nb N) helps in improving the biocompatibility of the stent 100 and reduces cracking of first and second struts 150a and 150 of the stent 100 while deploying the stent 100 in the tortuous anatomy of the blood vessel. Further, it acts as a protective layer on the stent 100 surface thereby reducing the risk of blood clotting during blood flow. The coating does not affect any material properties and/or any other biomechanical functionality of the stent 100. The long term chemical stability of material results in higher adhesive strength between the coating and the surface of the stent 100.

In an alternate embodiment, as shown in FIG. 4, the present invention discloses a tapered stent 600. The tapered stent 600 is similar to the stent 100 but includes an axially tapered profile. For example, similar to stent 100, the tapered stent 100 includes a proximal end 610, a distal end 620, struts 650, rings 660, links 670 forming rows of open cells 630 and closed cells 640, etc. The tapered stent 600 may be used at tapered peripheral body lumens for treatment of any peripheral artery disease. The hybrid cell structure of the tapered stent 600 provides excellent positioning of the stent 100 in tapered peripheral target sites.

In an exemplary embodiment, in an expanded state, the tapered stent 600 includes a tapered diameter that is relatively larger at a proximal end 610 than a distal end 620 with continuous and gradual increase in the tapered diameter from the proximal end 610 to the distal end 620. The tapered profile of the tapered stent 600 provides excellent radial strength and support to the inner lumen of the artery without any displacement. The tapered stent 600 provides adequate strength to the long blood vessels which are non-parallel. The tapered diameter of the tapered stent 600 tapers from 0.3 mm to 4 mm and more preferably 0.5 mm to 0.75 mm, without affecting the radial strength of the tapered stent 600.

In an exemplary embodiment, the tapered stent 600 tapers from 0.55 mm to 0.3 mm, 0.8 mm to 0.55 mm, 1.05 mm to 0.8 mm, 1.3 mm to 1.05 mm, 1.55 mm to 1.3 mm, 1.8 mm to 1.55 mm, 2.05 mm to 1.8 mm, 2.3 mm to 2.05 mm, 2.55 mm to 2.3 mm, 2.75 mm to 2.55 mm, 2.75 mm to 2.25 mm, 3 mm to 2.5 mm, 3.5 mm to 2.75 mm, 3.5 mm to 3 mm, 4 mm to 3.5 mm, 5 mm to 4.5 mm, 5.5 mm to 5 mm, 6 mm to 5.5 mm, 6.5 mm to 6 mm, 7 mm to 6.5 mm, 7.5 mm to 7 mm, 8 mm to 7.5 mm, 2 mm to 1.25 mm, 3 mm to 2.30 mm, 4 mm to 3.35 mm, 5 mm to 4.40 mm, 6 mm to 5.45 mm, 7 mm to 6.25 mm, 8 mm to 7.25 mm or a combination thereof. The tapered stent 600 may have a predefined length ‘L’ according to the application/requirement. The predefined length ‘L’ of the tapered stent 600 ranges from 20 mm to 80 mm, more preferably from 30 mm to 60 mm.

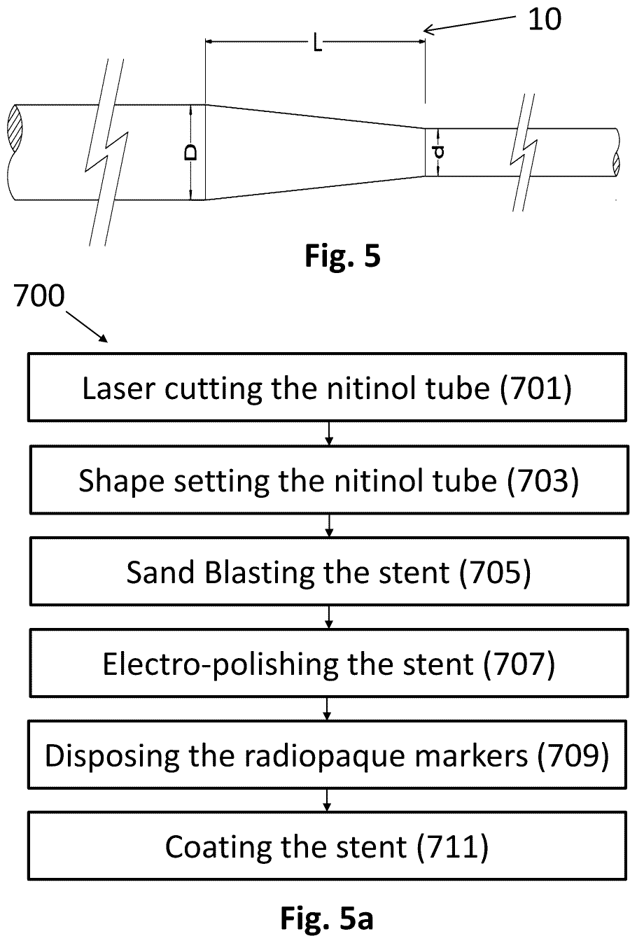

In an exemplary embodiment, a tapered mandrel 10 (as depicted in FIG. 5) is used to manipulate the Nitinol tube to yield the tapered stent 600. Similarly, in an alternate embodiment, a non-tapered mandrel (not shown) is used to manipulate the Nitinol tube to yield the stent 100. The Nitinol tube used to manufacture the stent 100 (or tapered stent 600) may be processed with respect to variation in a design, a wall thickness and/or a width of the Nitinol tube to achieve sufficient desired properties as per requirement for stenting. The mechanical properties (such as flexibility) of the stent 100 (or tapered stent 600) may depend upon the wall thickness and the hybrid cell structure. The wall thickness of the Nitinol tube ranges from 150 μm to 400 μm, and most preferably around 250 μm to 350 μm. A pre-defined diameter of the Nitinol tube may range from 1.2 mm to 3 mm, most preferably from 1.4 mm to 2.5 mm for stent 100.

The Nitinol tube used to make the tapered stent 600 may have a diameter that tapers from 0.5 mm to 2.2 mm, more preferably from 0.6 mm to 2 mm. It should be noted that the nitinol tube may be allowed to expand during a heat setting process to yield the stent 100 (or tapered stent 600). Hence, the diameter of the stent 100 (or tapered stent 600) is relatively more than the Nitinol tube.

FIG. 5a depicts an exemplary method 700 to make the tapered stent 600 of the present invention. The method begins at step 701, where the nitinol tube may be laser cut to form a pre-defined pattern using a laser. The laser may have one or more parameters including but not limited to argon pressure, water pressure, laser beam cutting speed, power, frequency, etc. The parameters of the laser may be adjusted according to requirement. In an exemplary embodiment, the nitinol tube is laser cut to yield the hybrid cell structure of the tapered stent 600.

Additionally or optionally, the laser cut nitinol tube may be processed to remove any abnormalities arising from laser cutting the nitinol tube. In an exemplary embodiment, the laser cut nitinol tube is processed via grinding and honing processes.

At step 703, the laser cut nitinol tube may be heat treated (or annealed) with a shape setting process on the tapered mandrel 10 to yield a tapered profile of the tapered stent 600. The tapered mandrel 10 may have a minimum diameter ‘d’ ranging from 2 mm to 8 mm. The tapered mandrel 10 may have a maximum diameter ‘D’ ranging from 2.5 mm to 8.5 mm. The dimensions of the nitinol tube and the tapered mandrel 10 depend upon the required dimensions of the tapered stent 600. The tapered mandrel 10 may have a taper angle which influences the gradual tapering of the tapered stent 600. The taper angle may be derived from an equation, tan φ=D−d/2 L; where φ is the taper angle with respect to a center of the mandrel 10.

The laser cut nitinol tube may be annealed over the mandrel 10 at a predefined temperature thereby, permanently deforming the Nitinol tube corresponding to the mandrel 10. The predefined temperature ranges from 450° C. to 550° C. In an exemplary embodiment, the laser cut nitinol tube is annealed at 500±5° C. to yield the tapered stent 600.

At step 705, the tapered stent 600 may be sand blasted to maintain the uniformity of the tapered stent 600. Sand blasting provides a smooth surface finish to the tapered stent 600. Further, sand blasting thoroughly cleans and removes unwanted burrs from the surface of the tapered stent 600 thereby removing dirt and sharp edges from corners and surface of the tapered stent 600.

In an optional step 707, a high grade of electro-polishing and/or surface finishing may be provided over the tapered stent 600 which increases durability of the tapered stent 600.

At step 709, the radiopaque markers 190 may be irreversibly coupled to the proximal end 610 and distal end 620 of the tapered stent 600 (described above).

At step 711, the tapered stent 600 may be coated with the pre-defined coating material thus yielding the one or more coating layer over the tapered stent 600. In an exemplary embodiment, the tapered stent 600 is coated inside a vacuum chamber with the help of arc PVD method. The tapered stent 600 is continuously rotated with the help of a rotating device for a pre-defined time period inside the vacuum chamber. The pre-defined coating material is washed with an alkaline solution for at least 4 minutes at a temperature ranging from 60° C. to 70° C. The alkaline solution may have a pH ranging from 9.0 to 10.0, more preferably 9.5 to 9.8. Thereafter, the coated tapered stent 600 is rinsed under distilled water and air dried in a cabinet at 70° C. The coated tapered stent 600 may be tested for coating integrity on the basis of one or more acceptance criteria. The acceptance criteria may include but not limited to smooth surface free from any coating defects such as rough surface, foreign particles, lumps, coating damage, etc.

The method 700 to make the tapered stent 600 can be used to make stent 100 by replacing the tapered mandrel 10 with the non-tapered mandrel.

After making the stent 100 (or tapered stent 600), the stent 100 (or tapered 600) may be inspected for quality. No mechanical damage was observed in the stent 100 during and after extensive compressive loading of the stent 100, thereby indicating that the stent 100 perfectly satisfies the criteria for local compression test and crush resistance test. The stent 100 also passed the delivery deployment and retraction test without any resistance, kinking, bending, fracture or any other visual damage to the stent 100 and a delivery system.

The stent 100 may require a force for deployment. The force may be less than 25 N. In an exemplary embodiment, the force required to deploy the stent 100 having diameter of 5 mm and length of 30 mm is in the range of 9 N to 11.5 N. In an exemplary embodiment, the force required to deploy the stent 100 having diameter of 10 mm and length of 120 mm is in the range of 17 N to 20 N.

In an exemplary embodiment, the stent 100 have a kink radius of 2 mm for the entire length of the stent 100. Hence, the stent 100 have excellent flexibility and kink resistance.

Radial strength of the stent 100 may be represented as radial force. In an exemplary embodiment, the radial force of the stent 100 having diameter of 5 mm and length of 30 mm is in the range of 613.64 mmHg to 663.66 mmHg. In an exemplary embodiment, the radial force of the stent 100 having diameter of 10 mm and length of 30 mm is in the range of 164.95 mmHg to 175.88 mmHg. Hence, the stent 100 have excellent radial force to safely withstand physiological conditions of the target site.

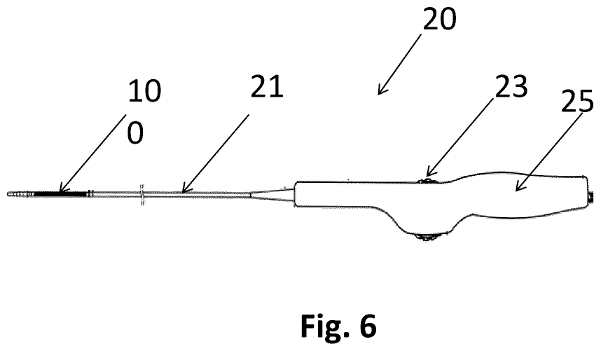

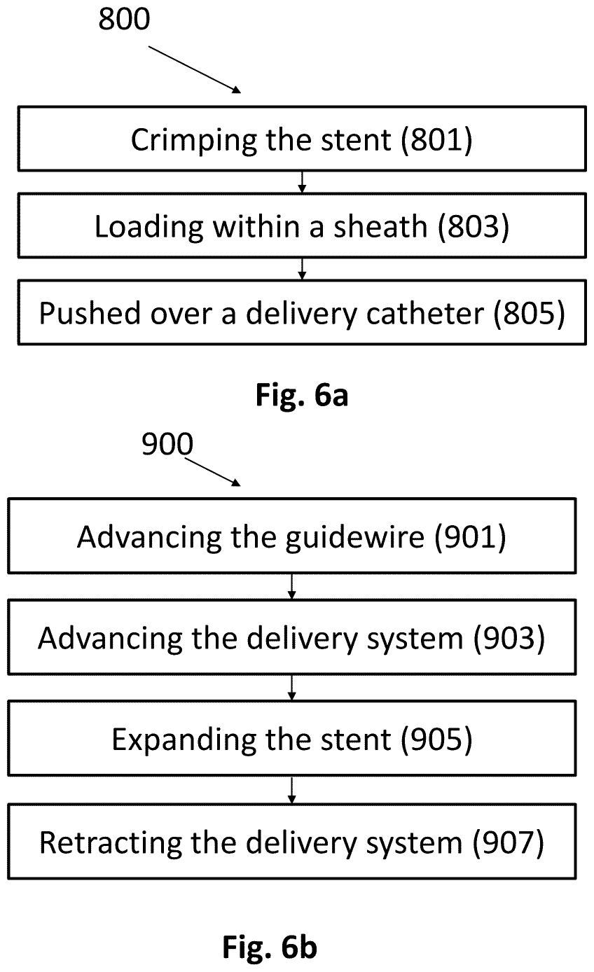

FIG. 6 shows an exemplary delivery system 20 that may be used to deploy the stents 100 at the target site. The stent 100 may be pre-loaded on a delivery catheter 21 of the delivery system 20 in the collapsed state and further constrained by a sheath (not shown) to prevent self-expansion.

FIG. 6a depicts an exemplary method 800 to pre-load the stent 100 on the delivery system 20. Proper pre-loading of the stent 100 is vital for uniform loading and deployment of the stent 100 without any damage. In an exemplary embodiment, the stent 100 having length up to 135 cm is used with 5 French catheter delivery system 20. The delivery system 20 may have a length including 80 cm, 120 cm, 135 cm, etc. The delivery system 20 may have one or more radiopaque marker (not shown) on the delivery catheter 21 of the delivery system 20 to help positioning of the delivery system 20. In an exemplary embodiment, the delivery system 20 is a braided pullback delivery system to provide optimal push ability and excellent track-ability. At first step 801, the stent 100 may be crimped from the expanded state to a collapsed state by any crimping mechanism. In an exemplary embodiment, the stent 100 is crimped with a crimper.

At step 803, the stent 100 in its collapsed state may be loaded within a sheath. The sheath may have an inner diameter. The inner diameter of the sheath may depend upon the diameter of the stent 100 in its expanded state. In an exemplary embodiment, the sheath has a diameter of 5 French for stent 100 having a diameter of 5 mm to 10 mm in its expanded state.

At step 805, the stent 100 may be pushed over a delivery catheter 21 of the delivery system 20.

The method 800 may be followed to pre-load the tapered stent 600 as well.

FIG. 6b depicts an exemplary deployment method 900 for implanting the stent 100 at a target site. Additionally or optionally, before the method 900 begins, pre-dilation of the target site may be performed. The pre-dilation of the target site ensures effective deployment process.

At first step 901, a guide wire is advanced at a location of a lesion (the target site) through an introducer sheath (access site). In an exemplary embodiment, the guide wire has a diameter of 0.035 in. (0.89 mm). Advancement of guidewire before the delivery catheter 21 offers fewer traumas to the vessels and makes arterial sub selection easier. Arterial sub selection corresponds to inspection of the target site via angiography techniques and selecting appropriate dimension of the stent 100 to be implanted at the target site.

At step 903, the delivery system 20 may be advanced over the guidewire through a hemostatic valve of the introducer sheath as a single unit. The delivery system 20 may be advanced until it reaches a central location of the target site. A position of the delivery system 20 may be verified by inspecting the radiopaque marker of the delivery system 20 under fluoroscopy.

At step 905, the sheath of the delivery system 20 may be pulled back in relation to the delivery catheter 21 of the delivery system 20 thereby facilitating expansion of the stent 100 from the collapsed state to the expanded state. The sheath of the delivery system 20 may be pulled back by rotating a thumb wheel 23 disposed on a handle 25 of the delivery system 20. Once the radiopaque markers 190 disposed at the distal end 120 of the stent 100 starts separating, the deployment of stent 100 is confirmed to have started. The position of the proximal end 110 and distal end 120 of the stent 100 may be verified by inspecting the radiopaque markers 190 in relation to the target site under fluoroscopy.

After the distal end 120 of the stent 100 is perfectly positioned at the target site, the thumb wheel 23 can further be rotated until the entire length of the stent 100 is being expanded. The thumb wheel 23 may be rotated until the radiopaque markers 190 at the proximal end 110 of the stent 100 separates similar to the distal end 120.

Before separation of the radiopaque markers 190 at the proximal end 110 of the stent 100, the stent 100 may be re-sheathed within the sheath of the delivery system 20 to reposition the stent 100 in relation to the target site.

At step 907, the delivery system 20 may be retracted from within the stent 100 and the target site. The deployed stent 100 improves luminal diameter at the target site by physically supporting narrowed peripheral vessels and restores blood flow.

The method 900 may be followed to implant the tapered stent 600 at the target site as well.

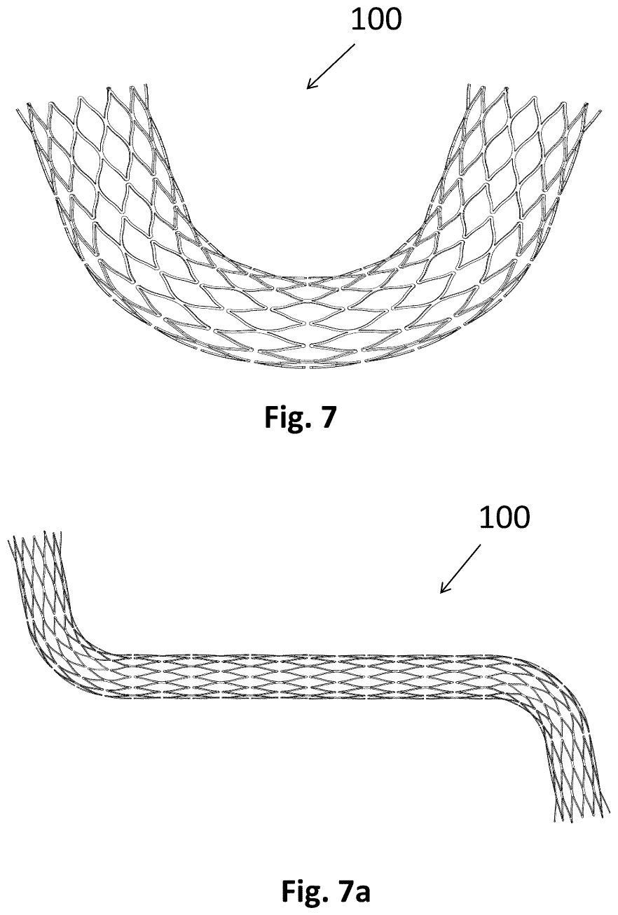

The stent 100 (or tapered stent 600) may conform to the anatomy of the target site after deployment. In an exemplary embodiment, as shown in FIG. 7, the stent 100 forms a C-shape corresponding to the anatomy of the target site post implantation. In another exemplary embodiment, as shown in FIG. 7a, the stent 100 forms an S-shape corresponding to the anatomy of the target site post implantation. The hybrid cell structure of the stent 100 (or tapered stent 600) ensures integrity of the stent 100 (or tapered stent 600) irrespective of the anatomy of the target site thereby significantly reducing post-implantation complications.

The present invention is now described with the help of the following examples:

Example 1 (Prior art): A Nitinol tube having a diameter of 3 mm and thickness of 300 μm was laser cut to make a stent of 5 mm diameter and 30 mm in length. The stent was completely made of closed cell structure. Such stent structure resulted in high radiopaque properties but had low flexibility owing to the closed cell profile.

The design of stent included closed cells throughout the length by interconnecting the closed cells having four to five straight interlink / connecting link with fillet edges at both ends of the connector's links. The stent after manufacturing was loaded onto the delivery catheter. Thereafter, a bench test was carried out to check the feasibility of the stent. The bench test results of the closed cell stent revealed that the stent broke from various joints due to the rigid profile. It had poor mechanical properties for most of the stenting applications.

Example 2 (Present invention): A Nitinol tube having diameter of 3 mm and a thickness of 300 mm was laser cut to yield the stent 100 of the present invention. The stent 100 had a diameter of 5 mm and a length of 30 mm. The laser cut stent 100 was shape set to relieve stress and obtain the desired shape of the stent 100. Thereafter, the stent 100 was electro polished to get a smooth and shiny surface. Further, a coating of Titanium-Niobium Nitride (Ti-Nb N) was coated in an inert environment (nitrogen gas) to improve adherence of the coating uniformly on to the stent's 100 surface. The stent 100 was then loaded into 5 French delivery catheter 21 for deployment at the target site. The stent 100 loaded into the catheter was further sterilized using EtO gas and packed in Tyvek pouch and an outer secondary box.

The deployment force required to deploy the stent 100 at the target site was not more than 25 N. The coating on the stent 100 reduced the chances of corrosion and thrombus formation at the lesion of the patient's body after implantation. The stent 100 was found to have a radial force of 638.42 mmHg which was suitable for deployment at the target site. The stent 100 was found to grip the inner lumen of the vessel after deployment without any displacement, resulting in increased holding capacity and resistance to migration of the stent 100.

Example 3 (Present invention): A Nitinol tube having diameter of 3 mm and a thickness of 300 mm was laser cut to yield the tapered stent 600 of the present invention. The diameter and length of the resultant tapered stent 600 was 5 mm and 30 mm respectively. The tapered stent 600 was placed in a tapering peripheral arterial lumen of the patient's body. The tapered profile of the tapered stent 600 gave excellent radial strength and support to the artery's inner lumen without causing any displacement. The tapered stent 600 had a tapering diameter in the expanded state from 6 mm to 5.25 mm. The tapered stent 600 had a length of 60 mm. The hybrid cell structure provided excellent positioning of the tapered stent 600 in the tapered peripheral target sites. The tapered shape of the tapered stent 600 was achieved using a corresponding mandrel 10.Thereafter, the tapered stent 600 was electro polished to get a smooth and shiny surface. Further, a coating of Titanium-Niobium Nitride (Ti-Nb N) was coated in an inert environment (nitrogen gas) to improve adherence of the coating uniformly on to the tapered stent's 600 surface. The tapered stent 600 was then loaded into 5 French delivery catheter 21 for deployment at the target site. The tapered stent 600 loaded into the catheter was further sterilized using EtO gas and packed in Tyvek pouch and an outer secondary box.

The deployment force required to deploy the tapered stent 600 at the target site was not more than 24 N. The coating on the tapered stent 600 reduced the chances of corrosion and thrombus formation at the lesion of the patient's body after implantation. The tapered stent 600 was found to have a radial force of 540.20 mmHg which was suitable for deployment at the target site.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim

1. A stent (100) to be deployed in a lumen with reduced risk of displacement, the stent (100) comprising:
a proximal end (110) and a distal end (120);
a proximal most ring (160a) disposed at the proximal end (110) and a distal most ring (160b) disposed at the distal end (120), the proximal most ring (160a) and the distal most ring (160b) include a plurality of first struts (150a) connected to each other in a predefined pattern forming a crown (195) at each connection;
a plurality of rings (160) extending coaxially between the proximal most ring (160a) and the distal most ring (160b), each of the plurality of rings (160) include a plurality of second struts (150) connected to each other in the predefined pattern forming the crown (195) at each connection;
a first link (170a) coupling a crown (195) of one of the proximal most ring (160a) or the distal most ring (160b) to a respective adjacent crown (195) of an adjacent ring (160);
a second link (170) coupling a crown (195) of a ring (160) to a respective adjacent crown (195) of an adjacent ring (160);
two or more rows of closed cells (130) disposed at each of the proximal end (110) and the distal end (120) respectively, each row of closed cells (130) formed by coupling each crown (195) formed by at least one of the plurality of second struts (150) to a respective adjacent crown (195) formed by at least one of the plurality of first struts (150a) or the plurality of second struts (150) via at least one of the first links (170a) or the second links (170);
a plurality of rows of open cells (140) flanked by two or more rows of closed cells (130), each row of open cells (140) formed by selectively coupling a crown (195) formed by at least one of the plurality of second struts (150) to a respective adjacent crown (195) formed by the plurality of second struts (150) via the second links (170) the second links (170) of the plurality of row of open cells (140) are arranged in a predefined pattern;
a plurality of radiopaque markers (190) coupled to at least one of the crowns (195) of the proximal most ring (160a) and the distal most ring (160b), the plurality of radiopaque markers (190) coupled to the proximal most ring (160a) are circumferentially offset from the plurality of radiopaque markers (190) coupled to the distal most ring (160b);
wherein, the plurality of first struts (150a) is longer than the plurality of second struts (150);
wherein, the first link (170a) is offset inwardly to enable the plurality of first struts (150a) of the proximal most ring (160a) and the distal most ring (160b) of the stent (100) to flare by a pre-defined angle (x) relative to a central axis at the proximal end (110) and the distal end (120) respectively, in an expanded state of the stent (100), thereby reducing risk of any displacement when the stent (100) is deployed in a lumen.

2. The stent (100) as claimed in claim 1, wherein the stent (100) includes a uniform diameter.

3. The stent (100) as claimed in claim 1, wherein the stent (100) is axially tapered to yield a tapered stent (600).

4. The stent (100) as claimed in claim 1, wherein the plurality of first struts (150a) and the second struts (150) is connected in a zig-zag pattern to form the crowns (195) at each connection.

5. The stent (100) as claimed in claim 1, wherein the plurality of crowns (195) is relatively wider compared to the first struts (150a) and the second struts (150).

6. The stent (100) as claimed in claim 1, wherein the first link (170a) and the second link (170) is straight shaped.

7. The stent (100) as claimed in claim 1, wherein the first link (170a) and the second link (170) is S-shaped.

8. The stent (100) as claimed in claim 1, wherein the pre-defined pattern of the second links (170) include a staggered pattern (170b).

9. The stent (100) as claimed in claim 1, wherein the pre-defined angle (x) ranges from 160 to 180 degrees.

10. The stent (100) as claimed in claim 1, wherein the radiopaque marker (190) is welded to at least one of the crowns (195) of the proximal most ring (160a) and the distal most ring (160b).

11. The stent (100) as claimed in claim 1, wherein the stent (100) is coated with a pre-defined coating material.

12. The stent (100) as claimed in claim 1 or 11, wherein the stent (100) is coated with at least one of an anti-proliferative drug and/or a thin layer of Titanium-Niobium Nitride.
